(19) United States
(12) Patent Application Publication
(10) Pub. No.: US 2007/0179360 A1
(43) Pub. Date: Aug. 2, 2007

(54) APPARATUS FOR MEASUREMENT OF BODY COMPOSITION

(76) Inventor: Richard P. Mikat, LaCrosse, WI (US)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 11/670,811

(22) Filed: Feb. 2, 2007

Related U.S. Application Data
(60) Provisional application No. 60/764,490, filed on Feb. 2, 2006.

(51) Int. Cl. A61B 5/00

(52) U.S. Cl. 600/300

(57) ABSTRACT

An apparatus for measurement of an individual's body composition includes a pressure mat configured to fully support an adult supine patient and to provide a series of pressure readings at a series of cells over the surface of the pressure mat and then electronically transmits the readings to a computer or the like. The pressure readings along with other relevant clinical information such as the patient's weight are then used to determine the patient's body composition such as percent body fat, body density, total body volume, fat mass and fat-free mass.

### CROSS REFERENCE TO RELATED APPLICATIONS

This application claims the benefit of U.S. provisional application 60/764,490 filed Feb. 2, 2006 hereby incorporated by reference.

### STATEMENT REGARDING FEDERALLY SPONSORED R & D

### BACKGROUND OF THE INVENTION

The present invention relates to devices to measure body composition, such as body fat, and in particular to a device measuring the pressure of the body against a surface to deduce body composition.

Accurate and reliable evaluations of a person's body composition, in particular, body fat percent, is useful in the assessment of a person's overall health and physical fitness. In addition, such assessments are useful in assisting individuals in managing their weight over a period of time. Excess body fat has been shown to increase a person's risk of developing numerous diseases and conditions such as coronary artery disease, cancer, hypertension, depression, impaired heat tolerance, hypercholesterolemia, and type II diabetes mellitus. Further, body composition plays a significant role in an individual's ability to participate in various physical activities such as exercise and various sporting activities.

Currently, there exist numerous methods of assessing an individual's body composition. Common laboratory techniques include Dual-Energy X-ray Absorbtiometry (DEXA), underwater weighing (hydrodensitometry), total body water methods, whole-body plethysmography and radiographic tomography (REFS). Of these, DEXA is considered by many in the relevant art to be the most reliable means of determining an individual's body composition. Common field techniques include anthropometry by skinfold and/or tape measurements, bioelectrical impedance and near-infrared reactance (REFS).

Typically, laboratory techniques are considered to be more reliable than field techniques. However, laboratory techniques are usually more time consuming, costly and difficult to administer and tolerate. Field techniques, while portable and less time consuming and expensive are considered less accurate and precise than laboratory techniques commonly employed. Some field techniques are as difficult to administer and tolerate as laboratory techniques.

Thus, it is desired to provide a method of determining an individual's body composition that does not suffer from the disadvantages of currently used methods. Namely, a method of determining an individual's body composition that is accurate, precise, cost-effective, time efficient, and easily administered and tolerated is desired.

### BRIEF SUMMARY OF THE INVENTION

The present inventor has recognized the need to provide an accurate, precise, cost-effective, time efficient and easily administered and tolerated method of determining an individual's body composition. Generally, the present invention contemplates the use of total-body pressure mapping for overcoming the inadequacies experienced by current methods of determining and individual's body composition.

Specifically then, the present invention provides a device for measuring body composition including a pressure sensor, such as an array of pressure sensitive transducers, providing a pressure map indicating pressures over a range of points of an area receiving a body being measured and a composition analyzer receiving pressure data from the pressure mat to output a body composition measurement.

Thus it is feature of at least one embodiment of the invention to provide volumetric analysis of a patient using a simple pressure mat. It is another object of the invention to provide body composition measurement without immersion of the patient or exposure of the patient ionizing radiation and without tester-based measurement errors common in current methods such as skinfold testing.

The composition analyzer may output body density, a proportion of body fat to non-fat tissue, or estimates of the fat mass fat-free mass, and body volume of the body based on the pressure map data.

Thus it is a feature of at least one embodiment of the invention to provide a body composition measuring instrument that may provide a range of different body composition measurements.

Furthermore, the composition analyzer of the present invention may include a model relating a pressure map to at least one of fat mass fat-free mass, percent body fat, body density, and body volume. In addition, the model may be a regression based on empirical data.

It is thus a feature of at least one embodiment of the invention to provide a simple method of equating readily determined body metrics to body composition.

The pressure map provided by the pressure sensors may be configured to cover substantially the entire body.

The composition analyzer of the present invention may be configured to partition the pressure maps into zones so as to relate each zone to a given body volume or composition measurement.

It is thus a feature of a least one embodiment of the invention to allow fine tuning of the technique, for example by using different empirical models, to different areas of the body.

The body being analyzed may be a human body where the area pressure sensor indicates the pressure when the human body lies supine on a horizontal surface.

It is thus another feature of at least one embodiment of the invention to provide a measurement technique that is comfortable and convenient for the user.

The composition analyzer may be configured to receive a series of pressure maps taken over a period of time in order to process the entire series of pressure maps thus reducing the variation caused by the breathing of the individual.

It is thus a feature of at least one embodiment of the invention to provide a body composition instrument that does not require the patient to hold his or her breath.

Various alternative embodiments and modifications to the invention will be made apparent to one of ordinary skill in the art by the following detailed description taken together with the drawings.

### BRIEF DESCRIPTION OF THE DRAWINGS

FIG. 1 is a simplified block diagram of the principal components of a preferred embodiment of the invention showing a pressure mat and an electronic scale communicating with a computer for analyzing the data from the pressure mat and scale;

FIG. 2 is a side elevational view of the mat of FIG. 1 with superimposed pressure data and an outline of a supine patient;

FIG. 3 is a chart showing a binning of pressure data according to pressure ranges per one embodiment of the present invention;

FIG. 4 is a chart showing variations in pressure data as a function of time and alternative methods of removing variation caused by periodic patient motion; and

FIG. 5 is a simplified representation of a map of pressure data obtained by the pressure mat of FIG. 1 and showing division of the pressure map into zones for independent evaluation.

### DETAILED DESCRIPTION

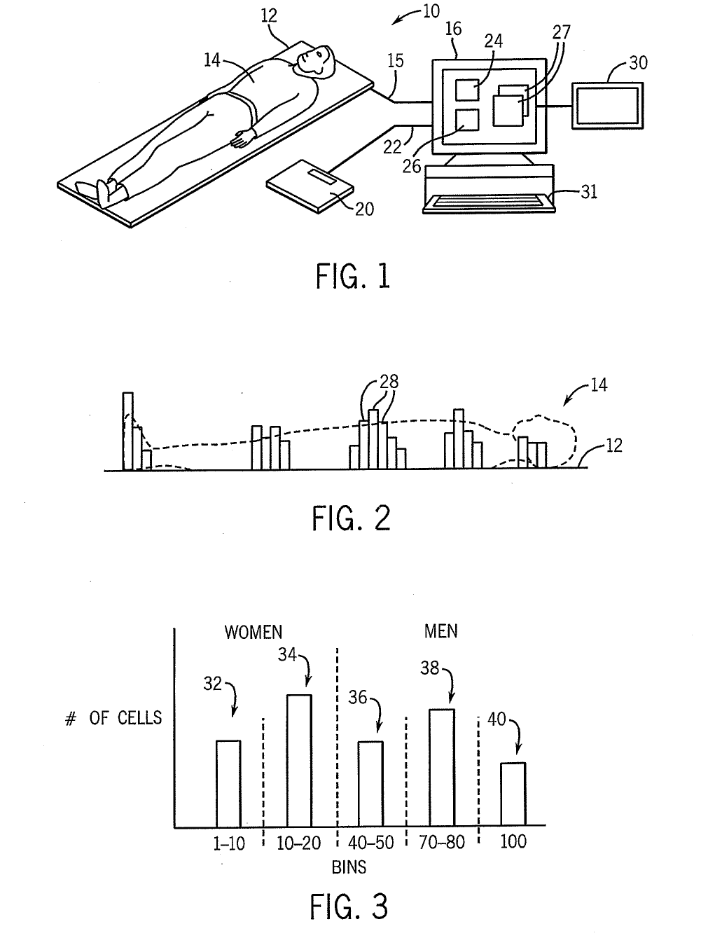

Referring now to FIG. 1, a body composition device 10 of the present invention may include a pressure mat 12 sized to fully support a supine patient 14. The pressure mat 12 provides a series of pressure readings, for example, in millimeters of mercury, at a series of cells, such as piezo-resistive transducers, over the surface of the pressure mat 12 and may provide them electronically through cable 15 to a computer 16 or the like.

Pressure mats of this kind are typically available from Force Sensitive Applications (FSA, model no. 477, Winnipeg, Manitoba, Canada). Typical dimensions of such mats are about 79 by 203 cm and contain about 1024 pressure-sensitive piezo-resistive transducers. Such pressure mats are typically calibrated to a pressure range of 0-100 mm Hg, having variation coefficients of less than 10%.

A standard clinical scale 20 may also be connected by cable 22 to the computer 16 to provide a weight of the individual 14 taken earlier or later.

The computer 16 may execute a stored program 24 as will be described, accepting as arguments, the data from the pressure mat 12 and the scale 20 and applying the data to one or more models 26 to produce an output report 27 such as may be printed and/or displayed on a computer screen or the like. The output report 27 may provide a measure of body composition of the patient 14 such as percent body fat (whole-body or regional), body density, total body volume, fat mass and fat-free mass.

Data entry apparatus such as a keyboard 31 may be provided for the manual entry of data, for example, patient height, age and sex, as well as, in an alternative embodiment, the patient's weight from the scale 20.

Referring to FIG. 2 generally, the pressure mat 12 will provide a series of pressure values 28 distributed over the map generally clustered in the locations of the patient's head, upper torso, hips, lower legs, and feet and hands.

An analysis of the pressure map comprising these pressure values 28, and optionally their locations, can be highly correlated to body composition. It is possible that the pressure values serve as a proxy of body volume or that the pressure values directly reflect relative amounts of body fat, though other theories are contemplated.

In a first embodiment, now to be described, the pressure values 28 are binned into pressure ranges as shown in FIG. 3, wherein each bin holds the number of pressure values, that is, the number of cells on the pressure mat 12, reporting a pressure with the bin range. Two bins 32 and 34, respectively, having ranges of 1-10 mm Hg, respectively, are used for women, and three bins 36, 38, and 40, having ranges 40-50 mm Hg, 70-80 mm Hg and 100 mm Hg, respectively, are used for men.

A regression analysis can be used to relate the counts in each bin to a measure of body composition, for example: fat mass, as measured by another technique, for example, DEXA, hydrostatic displacement, or the like well known in the art. The number and ranges of the particular bins may be determined empirically by evaluating the correlation coefficients obtained with regressions using different binning.

The resulting regression formulas provide models that may be used to determine body composition.

At present, the preferred formula for predicting fat mass for women is as follows:

FM(g)=5163.577+(593.901*(1-10 mm. bin))+(−270.813*(10-20 mm. bin)).

For men, the formula is as follows:

FM(g)=−12008.0+(1761.114*(40-50 mm bin))+(1477.527*(70-80 mm bin))+(668.739*(100 or greater mm bin)).

Where FM(g) is the fat mass in grams.

Fat free mass may then be calculated by subtracting the fat mass from the total body mass determined from the scale 20. Percent fat mass may be determined to provide an indication of relative proportions of fat and non-fat tissue.

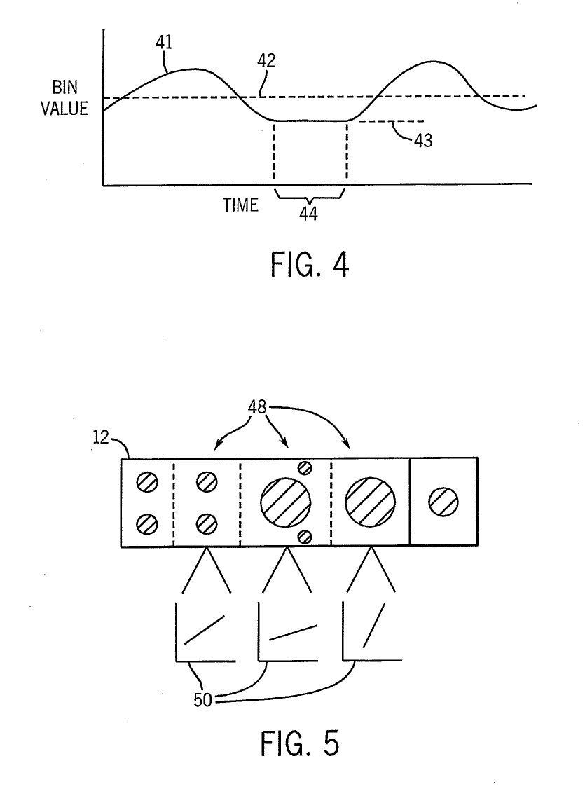

Referring now to FIG. 4, the pressure values 29 obtained from the pressure mat 12 may vary with time, for example, because of movement caused by breathing. These variations, reflected in any deduced quantity 41 (such as fat mass) introduce error into the determination of body composition. Several techniques may be used to reduce there errors including obtaining an average 42 of the quantity 41 over a period of time or for the purpose of breath motion, having the patient perform a breath hold during period 44, for example, at their functional residual capacity, a point at the base of the normal breathing cycle. An operator may instruct the patient in this breath hold and provide a trigger input to the body composition device 10 initiating the acquisition of data. Measurements during period 44 or the average 42 may be used in the generation of an output report 27.

Referring now to FIG. 5, the pressure values 28 obtained from pressure mat 12 are associated with location coordinates relative to area of the pressure mat 12. These location values allow the pressure values to be divided into a series of zones 48 (segment-zones), for example, dividing the head, upper torso, hips, lower legs, and feet and hands. Each of these zones 48, or groups of zones 48, may be separately modeled by regressions 50, which may be combined to provide accuracy in determination of body composition. The zones 48 may be fixed locations on the pressure mat 12 or preferably may be identified by the program 24 reviewing the pressure data and performing a pattern recognition process to identify particular portions of the patient, for example, by scaling and correlating a standard pressure map with pre-established zones. The operator may review this automatic determination of zones to confirm their placement and make any necessary adjustments. Certain zones such as the head or feet may be eliminated from the measurement process, and other zones reported out separately or combined using different weights resulting from different modeling regressions.

It will be understood from this description, that other mathematical modeling techniques may be used to relate pressure values 28 to body composition. Further, higher resolution analysis with smaller bins, or different bin ranges may be used. The spatial information obtained from the pressure mat 12 may also be used with pattern recognition techniques as may be empirically evaluated. Further, refined regressions and models may be developed for readily measured patient characteristics including patient age, weight, ethnicity, height, and the like, to provide improved accuracy.

Various alternatives are contemplated as being within the scope of the following claims particularly pointing out and distinctly claiming the subject matter regarded as the invention.

We hereby claim:

1. A device for measuring body composition comprising:
an area pressure sensor providing a pressure map indicating pressures over a range of points of an area receiving a body being measured; and
a composition analyzer receiving the pressure map to output a body composition measurement.

2. The device of claim 1 wherein the composition analyzer further receives a weight of the body being measured and wherein the body composition measurement output is body density.

3. The device of claim 1 wherein the body composition measurement output is at least one of fat mass, fat-free mass, percent body fat, body density, and body volume.

4. The device of claim 1 wherein the body being measured is a human body, and the pressure sensor provides a pressure map indicating pressures when the human body lies supine on a horizontal surface forming the area.

5. The device of claim 1 wherein the composition analyzer computes an estimate of at least one of fat mass, fat-free mass, percent body fat, body density, and body volume of the body based on the pressure map.

6. The device of claim 1 wherein the composition analyzer includes a model relating a pressure map to at least one of fat-free mass, percent body fat, body density, and body volume.

7. The device of claim 6 wherein the model is a regression relating empirical body composition measurements to the weight of the body being measured and the pressure map.

8. The device of claim 1 wherein the composition analyzer receives a series of pressure maps taken over time and processes the series to remove variation caused by breathing.

9. The device of claim 1 wherein the pressure map covers substantially an entire body of a human being.

10. The device of claim 1 wherein the composition analyzer partitions the pressure map into zones to separately relate each zone to a body composition.

11. A method of determining body composition comprising the steps of:
providing a pressure mat wherein a patient rests on the pressure mat in a supine position, such that a pressure map indicating pressures over a range of points of the patient are detected from the pressure being applied by the patient on the mat;
transmitting the pressure map to a computer;
calculating the patient's body composition by entering the pressure map and the patient's mass into an equation.

12. The method of determining body composition of claim 11 wherein a patient's gender is also transmitted to the computer and different equations are used for different genders.

13. The method of determining body composition of claim 11 further including the steps of weighing the patient to determine a patient's mass and wherein the patient's mass is transmitted to the computer wherein the patient's body density is calculated.

14. The method of determining body composition of claim 11 wherein the patient's proportion of body fat to non-fat tissue is calculated.

15. The method of determining body composition of claim 11 wherein a series of pressure maps are taken and transmitted to the computer so that the patient's body composition may be calculated based on a calculated average pressure map.

16. The method of determining body composition of claim 11 wherein an estimate of at least one of fat-free mass, percent body fat, body density, and body volume of the body is calculated based on the pressure map.

17. The method of determining body composition of claim 11 wherein the equation is an empirically derived model relating the pressure map to at least one of fat-free mass, percent body fat, body density, and body volume.

18. The method of determining body composition of claim 17 wherein the model relating the pressure map to fat mass is a regression of empirical data.

19. The method of determining body composition of claim 11 wherein the patient holds their breath during a measurement of the pressures of the pressure map to eliminate variation caused by the patient's breathing.

20. The method of determining body composition of claim 11 wherein the pressure map covers substantially the patient's entire body.

21. The method of determining body composition of claim 11 wherein the pressure map is partitioned into zones to separately relate each zone to a given body composition.
